(19) United States
(12) Patent Application Publication
(10) Pub. No.: US 2010/0305536 A1
(43) Pub. Date: Dec. 2, 2010

(54) ABSORBENT STRUCTURE

(75) Inventors: Maria Fernkvist, Molndal (SE); Anna Nihlstrand, Molndal (SE); Maryam Tondkar, Hisings Backa (SE); Shadi Ståhl, Molndal (SE); Malin Gullholm, Goteborg (SE)

Correspondence Address: BUCHANAN, INGERSOLL & ROONEY PC, POST OFFICE BOX 1404, ALEXANDRIA, VA, 22313-1404, US

(73) Assignee: SCA Hygiene Products AB, Goteborg (SE)

(21) Appl. No.: 12/667,910

(22) PCT Filed: Jul. 6, 2007
(86) PCT No.: PCT/SE07/50510

§ 371 Date: Jan. 27, 2010

(51) Int. Cl. A61F 13/53

(52) U.S. Cl. 604/372

(57) ABSTRACT

The invention relates to an absorbent structure (3) for use in an absorbent article (1) and being defined by an airlaid material structure (3) being formed by a plurality of layers (3a; 3b; 3c; 3d) which are interposed between a liquid-permeable top sheet (2) and an absorbent core (4), said absorbent article (1) also comprising a backsheet (5) being of liquid-impermeable material. According to the invention, the absorbent structure (3) comprises a first layer (3a) having resilient synthetic fibers of at least approximately 5 dtex; that the absorbent structure (3) comprises a second layer (3b) having superabsorbent particles generally arranged as a distinct layer as opposed to being integrated within any other material of said absorbent structure (3); and that the absorbent structure (3) also comprises a treated fluff material having hydrophilic properties.

### TECHNICAL FIELD

The present invention relates to an absorbent structure for use in an absorbent article and being defined by an airlaid material structure being formed by a plurality of layers which are interposed between a liquid-permeable top sheet and an absorbent core, said absorbent article also comprising a backsheet being of liquid-impermeable material.

### STATE OF THE ART

Today's absorbent articles, such as diapers, panty liners, incontinence pads, training pants and similar articles are manufactured by combining different types of material layers and absorbent bodies. A conventional absorbent article for use as an incontinence pad, for example, is normally designed as a layered product comprising a liquid-permeable top sheet (for example a nonwoven material based on synthetic or natural fibers) that faces the wearer's body, an airlaid fibrous material structure, an absorbent core, and finally a lower liquid-impermeable material, for example in the form of a thermoplastic backsheet that faces the wearer's clothing.

With regard to today's technology, there is a continuous demand for improvements of certain characteristics of absorbent articles. For example, it is important that such an article has optimum absorption properties. This means that the article must have a high degree of acquisition of fluid. Also, it must be designed with a low tendency for rewetting and it must also be perceived by the wearer as being more or less dry during use. These different properties of the absorbent article can be obtained to a varying degree by designing the article in a suitable manner as regards its dimensions and materials. It can be noted that there exists a number of airlaid structures having acceptable overall properties. These airlaid materials normally comprise a superabsorbent material, cellulose fibers and synthetic fibers of different length, thickness and having different types of treatment. However, with regard to prior art, it is difficult to obtain an article presenting all the above-mentioned properties at the same time.

With regard to prior art, the patent document EP 1356797 discloses a disposable absorbent article comprising an absorbent structure being composed of a number of distinct layers. More precisely, the absorbent structure comprises a first outermost layer having a mixture of fibers, an intermediate layer being constituted by bi-component fibers or some other suitable thermoplastic fibers, a fluid storage layer which may comprise a superabsorbent material, and finally a second outermost layer being composed of cellulose fibers.

An absorbent article designed in accordance with the EP 1356797 document presents an improved liquid handling and also increases the masking, i.e. the ability to hide stains due to absorbed liquid. However, as noted above, it is still difficult to obtain a high acquisition rate, a low rewet and a high sense of dryness at the same time.

Consequently, a problem with prior art is that it is difficult to improve absorbent articles so that all the above-mentioned properties (i.e. regarding liquid absorption, acquisition rate and preceived dryness) are present in an absorbent article of the type which comprises an airlaid absorbent structure.

### DISCLOSURE OF THE INVENTION

A primary object of the present invention is to provide an absorbent structure by means of which the above-mentioned problems and disadvantages can be overcome, in particular for providing an article such as an incontinence pad having excellent absorption, rewet and dryness properties.

This object is accomplished by means of an absorbent structure of the kind mentioned initially, wherein the absorbent structure comprises a first layer having resilient synthetic fibers of at least approximately 5 dtex, that the absorbent structure comprises a second layer having superabsorbent particles generally arranged as a distinct layer as opposed to being integrated within any other material of said absorbent structure, and that the absorbent structure also comprises a treated fluff material having hydrophilic properties.

By means of the invention, certain advantages will be obtained. Primarily, it can be noted that the invention leads to an absorbent structure having a very well balanced composition of the airlaid structure, which in turn contributes to a combination of certain advantageous properties. In particular, an optimized flow of liquid into the article is accomplished, due to a well-tuned design of the various layers forming part of the absorbent structure. In particular, each of the layers in the absorbent structure will have a suitable available volume to absorb incoming liquid and to distribute the liquid further into the other layers of the absorbent structure.

The invention consequently relies on the insight that by carefully selecting materials, properties and dimensions for the various materials in the absorbent structure, a more optimal and well-functioning balancing of the acquisition, rewet och dryness factors than previously known can be accomplished.

### BRIEF DESCRIPTION OF DRAWING

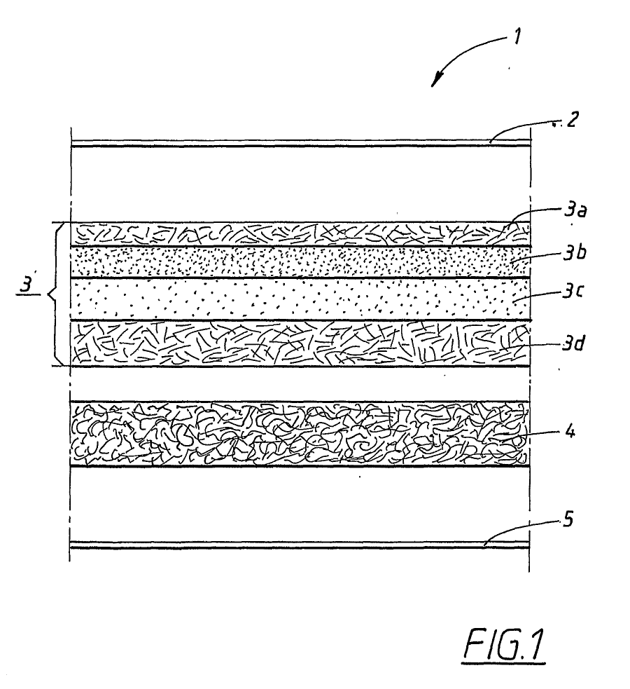

The invention will now be described with reference to a preferred embodiment and the appended drawing marked FIG. 1, which is a schematical cross-sectional view of an absorbent structure implemented in accordance with the present invention.

### PREFERRED EMBODIMENTS

The invention is suitably used in connection with absorbent articles, preferably in the form of relatively thin and compact incontinence pads. However, the invention is not limited to such absorbent articles only, but can in principle also be used in diapers, panty liners, incontinence articles, sanitary towels, training pants and similar articles. The appended drawing indicates a cross-sectional view of such an absorbent article 1. As indicated, the cross-section of the article 1 is designed in the form of a laminate with a number of sheets or layers. These layers are presented in a separated manner in the drawing.

Firstly, and in a conventional manner, the article 1 comprises a first layer in the form of a liquid-permeable topsheet 2, suitably a nonwoven material based on synthetic or natural fibers. The topsheet 2 is arranged to be closest to the body of the wearer of the article 1 during use thereof. Generally, the topsheet 2 is suitably manufactured from a nonwoven material being constituted by synthetic fibres such as polyethene, polypropylene, polyester, nylon or similar. Also, mixtures of different fibre types can be used for this purpose. Alternatively, the topsheet can also be made of other materials, such as a thermoplastic film, or a laminate or a combination of different laminates. Also, a nonwoven material sheet such as the topsheet 2 can be designed in various ways. For example, it can be perforated or non-perforated having a particular pattern.

Furthermore, an absorbent structure 3 in the form of a fibrous, layered airlaid material 3 is provided under the topsheet 2. This absorbent structure 3 is designed in accordance with the principles of the present invention and will be described in greater detail below. Furthermore, an absorbent core 4 of a generally known type is provided under the absorbent structure 3. Finally, the absorbent article 1 is also provided with a backsheet layer 5, preferably in the form of a liquid-impermeable thermoplastic material.

The absorbent core 4 is suitably manufactured from a fibrous material, in particular with natural or synthetic fibrers having absorbing properties. Alternatively, a mixture of natural and synthetic fibers, or other known absorbing materials, can be used. Furthermore, the absorbent core 4 may comprise a suitable amount of a superabsorbent material, for example 40-60% of the weight of the absorbent core 4. Obviously, such an arrangement will contribute to a very high absorption rate of the complete absorbent article 1.

The backsheet layer 5 is preferably liquid-impermeable (or has at least a very high resistance against penetration of liquid) and is consequently provided in order to prevent leakage of fluid out from the article 1. To this end, the backsheet layer 5 is suitably manufactured from a liquid-impermeable material such as a thin and sealing plastic film. For example, plastic films made from polyethene, polypropylene or polyester can be used for this purpose. Alternatively, a laminate of nonwoven material and a plastic film or other suitable material can be used as a backsheet layer 5.

Also, as previously known, the rear side of the backsheet 5 (i.e. the side of the backsheet layer 5 facing the garment of the wearer) can suitably be provided with one or more strips of adhesive or other attachment means which are used for attaching the article 1 onto a garment.

The principles of the present invention will now be described with reference to an embodiment of the invention. More precisely, the invention will be described with reference to an absorbent structure 3 to be used in an incontinence pad. As shown in FIG. 1, the absorbent structure 3 is based on an airlaid material which is preferably composed of four different layers. In the embodiment shown in FIG. 1, these four layers are indicated by reference numerals 3a, 3b, 3c and 3d. As will be described below, the absorbent structure 3 can alternatively also be composed of less than four distinct layers.

The first layer 3a is an upper layer in the sense that it is located closest to the topsheet 2. According to the embodiment, the first layer 3a comprises resilient, i.e. relatively stiff, synthetic fibers, with a dtex factor of at least 5 dtex, preferably more than 6 dtex, most preferably more than 16 dtex. Besides being rather stiff, these fibers have the function as binding fibers for the first layer 3a. The fact that these fibers have a relatively high dtex factor means that they will allow a relatively high intake rate of fluid into the article 1 and also that a relatively small intake zone is provided.

In addition to the resilient synthetic fibers, this first layer 3a preferably comprises a certain amount of liquid-absorbing, i.e. hydrophilic, fibers. Suitably, these fibers are constituted by a so-called treated fluff material, which is a material which is less hydrophilic than a conventional chemically based fluff fiber material. Also, according to known technology, such a treated fluff material is chemically treated to fiberize easily (i.e. more easily than a conventional non-treated fluff material) to yield a soft web which is more or less completely fiberized. An important feature of the treated fluff material is consequently that it is normally less hydrophilic than the conventional non-treated materials.

According to the embodiment, the treated fluff material is mixed with the resilient synthetic fibers mentioned above. The reason for adding such a material is that it leads to a product which is perceived as very dry and which has a smaller wet inlet area for the fluid being absorbed by the article. The proportion of the treated fluff material as compared with the total amount of material in the first layer 3a may vary within the scope of the invention, but is preferably 20-100%, and most preferably 50-100% of the total amount of material.

Furthermore, according to the embodiment, the second layer 3b of the absorbent structure 3 comprises a SAP material, i.e. superabsorbent particles. The superabsorbent material is preferably arranged as a distinct layer, i.e. as opposed to being arranged in the form of particles being mixed with, and therefore generally integrated with, any of the adjacent material layers (i.e. the first layer 3a and the third layer 3c).

By using superabsorbent particles as the second layer 3b, there is provided a high degree of dryness and a barrier against rewet of the surface of the absorbent article 1. In order words, liquid which has been absorbed by the article 1 is effectively prevented from returning from the absorbent core 4 and back to the topsheet 2.

The type and amount of superabsorbent particles to be used for the second layer 3b are crucial, and furthermore depend on the type of article in which the particles are to be used. Consequently, the exact type and amount of superabsorbent material may vary within the scope of the invention. For example, properties such as the absorption capacity, the acquisition time and the surface treatment of the superabsorbent particles vary between different brands and can consequently be used to optimize the performance of the product in question.

As examples only, the invention can suitably be implemented with a superabsorbent material having a weight of approximately 80-100 gsm when the absorbent article is a light and compact incontinence product, and can be implemented with a superabsorbent material having a weight of approximately 130 gsm when the absorbent article is a more heavy incontinence article.

Also, as a general rule, the amount and quality of the superabsorbent material should be adapted to the magnitude of the expected liquid volume to be absorbed by the article 1.

Furthermore, the third layer 3c of the absorbent structure 3 is a mixture of a chemical fluff material and synthetic binding fibres, preferably manufactured from polypropylene/polyethylene (PP/PE). Suitably, this latter material should be chosen with a low dtex value, preferably not more than 3.3 dtex.

A material such as the third layer 3c as described above will function so as to absorb the fluid in an effective manner and distribute it towards the absorbent core 4. It has even more hydrophilic properties than a treated fluff material and a more dense network of the fibres due to a lower dtex factor (i.e. a lower stiffness or resiliency) of the binding fiber as compared with the topsheet. Such properties will contribute to a high degree of dryness of the article 1.

The amount of binding fibres should be sufficient for the third layer 3c to stay intact as a distinct layer. However, the third layer 3c should not contain too much binding fibres since this leads to a situation where there will be an insufficient amount of absorbing fibres which can be used to drain the first layer 3a and the second layer 3b in order to keep the surface of the article 1 dry. According to the embodiment, the binding fibre should preferably be present in the third layer 3c with a percentage of approximately 20-50%.

Finally, the fourth layer 3d is constituted by a mixture of treated fluff material and binding fibres PE/PET (2.2 dtex). This means that this fourth layer 3d will function as a barrier against rewetting.

An absorbent article 1 having an absorbent structure 3 as described above will present certain advantageous properties. Firstly, it can be noted that it will have a high absorption rate. The topsheet material 2 will be drawn towards the airlaid structure 3 and there will be a good contact so that fluid will be transported quickly away from the topsheet 2 and towards the interior of the absorbent core 4 in order to be drained away. As a result, the absorbent article in question will be perceived as dry by the user. In summary, an article comprising an absorbent structure 3 according to the invention will have an even, undisturbed and relatively high flow of liquid into the interior of the article.

Also, the pore gradient of an article according to the invention—which contributes to the introduction of liquid into the underlying layers—is advantageous in the sense that it leads to an improved intake and a low degree of rewet. In particular, the pore gradient can be controlled by arranging the pores of the articles so that they are more and more dense for each layer of the absorbent article. Also, the hydrophilicity of each layer in the absorbent structure 3, i.e. the tendency of a material to be solvated by water, can be gradually increased for each layer down into the absorbent structure 3.

As described above, the invention can be used to implement an airlaid material which is well-balanced in the sense that it leads to optimal properties as regards liquid-absorption, rewet and dryness within the same article. However, the invention is not limited to the embodiment described above but can be varied. In the following, a few practical test results will be discussed in order to explain alternative solutions within the scope of the invention.

Firstly, it can be noted that practical tests have shown that the presence of absorbent and hydrophilic fibres, i.e. treated fluff material, in the first layer 3a is of great importance for the perception of dryness of the absorbent article 1 from the wearer's point of view. In particular, tests have shown that the presence of treated fluff material in the first layer 3a gives a higher sense of dryness for the wearer as compared with the case in which no treated fluff material is used, and also as compared with a conventional absorbent article according to prior art.

Furthermore, tests have also been carried out in which the rewet properties of the absorbent article 1 are shown to be clearly influenced by the amount of superabsorbent material in the second layer 3. Also, the properties of the absorbent article 1 are influenced by the design of the superabsorbent material, more precisely whether the superabsorbent material is arranged as a distinct layer (i.e. as preferred according to the invention) or mixed with the adjacent material layers, i.e. the first layer 3a and the third layer 3c. Accordingly, tests have shown that the above described embodiment involving a separate, distinct layer of superabsorbent material (i.e. the second layer 3b) leads to clearly improved rewet properties compared with prior art.

Tests have also been carried out which show that the amount of superabsorbent material also have influence on the rewet properties of an absorbent article designed in accordance with the invention. Accordingly, it has been noted that in general, the highest amount of superabsorbent material, being provided in the form of the second layer 3b, will give the lowest degree of rewetting of the entire absorbent article 1.

Furthermore, tests have been carried out in order to ascertain whether the dtex factor of the resilient synthetic fibers of the first layer 3a influences the acquisition rate. According to tests carried out, it has been demonstrated that a higher dtex rate corresponds to a relatively fast acquisition rate, as compared with a corresponding article but with a lower dtex rate.

Finally, tests have been carried out in order to ascertain whether the hydrophilic fibers in the first layer 3a should be treated or not. Such rewetting tests showed that the non-treated and treated fluff material present more or less the same rewet properties. However, tests of the non-treated fluff material gives a much larger wet area, which affects the perception of dryness in a quite negative manner. This means that the treated material is preferred according to the principles of this invention.

In summary, the basic principles of the invention relates to the following basic design measures. Firstly, the first layer 3a should comprise resilient synthetic fibres of at least approximately 5 dtex, and preferably also a treated fluff material having hydrophilic properties. Also, it should be noted that the treated fluff material can be present in the fourth layer 3d as described above. Also, the absorbent structure 3 should comprise a distinct layer of superabsorbent material, suitably in the form of the second layer 3b as described above.

In particular, it should be noted that it is beneficial with a treated fluff material in the first layer 3a in the case where the topsheet 2 is constituted by a material having a relatively dense structure. In such a case, there is a risk that the liquid will remain in the topsheet 2, with the consequence that the article 1 is perceived by the wearer as being undry. However, by using a treated fluff material in the first layer 3a, this layer will be more hydrophilic and slightly less resilient than if the first layer 3a only should contain synthetic fibers and no treated fluff material.

The invention is not limited to the embodiment described above but can be varied within the scope of the appended claims. For example, the absorbent article 1 as described is suitably an incontinence pad. However, the invention is not limited to such an article only but can be used for other absorbent articles.

In any case, the article is normally arranged to be manufactured by attaching the various layer to each other. Such manufacturing steps can be carried out by means of known measures, such as for example ultrasound welding and gluing. Such technology is previously known, and for this reason it is not described in detail here. The manufacturing process for the article suitably starts with the fourth layer 3d. After that, the third layer 3c is provided, then the second layer 3b and finally the first layer 3a.

The basic concept of the invention has been described with reference to an embodiment with four different layers. However, the invention can alternatively be implemented with a less number of layers. For example, the fourth layer 3d can in principle be omitted without departing from the general inventive principle. This is due to the fact that the advantageous properties of high acquisition, low rewet and high dryness are still accomplished to a satisfactory degree even if such a layer is not included.

Furthermore, the invention can be implemented using many types of materials and material combinations.

1. An absorbent structure for use in an absorbent article, the absorbent structure comprising an airlaid material structure formed by a plurality of layers which are interposed between a liquid-permeable top sheet and an absorbent core, said absorbent article further comprising a backsheet of liquid-impermeable material, wherein the absorbent structure comprises: (a) a first layer located closest of said layers to said topsheet and comprising resilient synthetic fibers of at least approximately 5 dtex; and (b) a second layer comprising superabsorbent particles generally arranged as a distinct layer as opposed to being integrated within any other material of said absorbent structure; wherein the first layer further comprises a certain amount of liquid-absorbing fibers constituted by a treated fluff material.

2. The absorbent structure according to claim 1, wherein the proportion of said treated fluff material as compared with the total amount of material in the first layer is 20-100% of the total amount of material.

3. The absorbent structure according to claim 2, wherein the proportion of said treated fluff material as compared with the total amount of material in the first layer is 50-100% of the total amount of material.

4. The absorbent structure according to claim 1, wherein the resilient synthetic fibers of the first layer is of at least approximately 16 dtex.

5. The absorbent structure according to claim 1, wherein the absorbent structure further comprises a third layer comprising a mixture of chemical fluff material and synthetic binding fibres.

6. The absorbent structure according to claim 5, wherein the synthetic binding fibres are present in the third layer with a percentage of approximately 20-50%.

7. The absorbent structure according to claim 1, wherein the absorbent structure further comprises a fourth layer comprising a mixture of treated fluff material and binding fibres PE/PET.

8. The absorbent structure according to claim 1, wherein the superabsorbent material has a weight of approximately 80-130 gsm.

9. An absorbent article (1) comprising the absorbent structure according to claim 1.

10. The absorbent structure according to claim 5, further comprising a fourth layer comprising a mixture of treated fluff material and binding fibres PE/PET.
